(19) United States
(12) Patent Application Publication
(10) Pub. No.: US 2013/0108571 A1
(43) Pub. Date: May 2, 2013

(54) NOVEL AGENT FOR IMPROVING THE DEPOSITION OF OIL ON HAIR

(71) Applicant: Monique Adamy, Paris (FR)

(72) Inventor: Monique Adamy, Paris (FR)

(73) Assignee: Rhodia Operations, Aubervilliers (FR)

(21) Appl. No.: 13/582,626

(22) PCT Filed: Mar. 3, 2011
(86) PCT No.: PCT/FR11/50442

§ 371 Date: Nov. 16, 2012

(30) Foreign Application Priority Data
Mar. 3, 2010 (FR) 10 51542

(51) Int. Cl. A61K 8/60; A61Q 5/00

(52) U.S. Cl. CPC A61K 8/604; A61Q 5/002; A61Q 5/00
USPC 424/70.12; 424/70.19; 424/70.13

(57) ABSTRACT

The use of an agent in a composition intended to be applied to hair is described. The composition can include an oil and a mixture including an amphoteric surfactant and an alkylpolyglucoside to increase a quantity of oil deposited on the hair.

The present invention relates to a novel agent intended for improving the deposition of oil on healthy or damaged hair.

It is known that hair which has been embrittled or damaged under the action of atmospheric elements (exposure to solar radiation, air pollution, wind, sea water, chlorinated water and the like) or under the action of mechanical treatments (such as brushing, combing, drying, rubbing against various supports) or chemical treatments (such as dyeing, bleaching and/or permanent waving, sleeking or straightening) is often difficult to disentangle or to style, has a rough feel, with no smoothness or shine. This appearance is in general correlated with a deterioration of the state of the cuticle, with separation, erosion or abrasion of the keratin scales, exposing the cortex to future attacks to a greater or lesser degree.

Accordingly, in order to improve the cosmetic properties of cleansing compositions intended to be applied to embrittled or damaged hair, it is now common to introduce into them conditioners whose main function is to limit or repair the harmful effects caused by the various treatments or attacks to which hair fibers are frequently subjected. These conditioners may also improve the cosmetic behavior of natural hair, in particular in terms of ease of disentanglement and combing. The conditioners most commonly used to date in detergent hair compositions are cationic or amphoteric polymers, silicones and/or silicone-containing derivatives, vegetable oils, which confer on wet or dry hair ease of disentanglement and combing, increased smoothness and sleeking, or even a marked improvement in shine compared with what may be obtained with the corresponding cleansing compositions not containing them. In these shampoos, commonly called “two-in-one”, it is preferably known to use a mixture of silicone oil and cationic or amphoteric polymer.

Current two-in-one shampoo formulations make it possible to obtain acceptable levels of oil, in particular silicone, deposits on natural hair. It is by contrast considerably more difficult to obtain substantial yields of deposition of oil, and more particularly of silicone, and more particularly of dimethicone, on damaged hair following chemical treatments such as dyeing, bleaching and/or permanent waving, sleeking or straightening. It is thus sought to improve the deposition of such oils. It is sought in particular to improve their deposition on hair damaged by chemical treatments, such as dyeing, bleaching and/or permanent waving, sleeking or straightening. Improving the deposition may make it possible in particular to optimize the quantities of oil formulated (by decreasing them). This may be economically advantageous.

The aim of the present invention is therefore to provide a novel agent, present in a shampoo, which makes it possible to improve the deposition of oil on healthy or damaged hair, during the application of said shampoo to hair.

Accordingly, the present invention relates to the use, in a composition, intended to be applied to hair, comprising an oil and a mixture consisting of an amphoteric surfactant and an alkyl polyglucoside, of said mixture in order to improve the deposition of said oil on hair.

In the present invention, and unless otherwise stated, the expression “oil” is understood to mean any hydrophobic liquid, immiscible with water.

In the present application, and unless otherwise indicated, the expression “improving the deposition of said oil on hair” is understood to mean increasing the quantities of oil deposited on the hair.

The composition used in the context of the present invention is intended to be applied to hair. This hair composition is preferably in the form of a shampoo. It preferably consists of so-called two-in-one shampoos which provide cleansing of the hair and conditioning.

Improvement of the deposition of oil is measured according to the following test:

Locks of natural brown hair (called “healthy hair” in the remainder of the present document) and of bleached hair (called “damaged hair” in the remainder of the present document) are used.

The protocol for evaluating the quantity of deposited silicone comprises four steps: pretreatment of the locks with a 10% solution of sodium lauryl ether sulfate active substance, treatment of the locks with the shampoo tested, silicone extraction with tetrahydrofuran (THF) and assaying of the extracted silicone by GPC with an ELSD detector (evaporative light scattering detector). The yield of silicone oil deposition on the hair is the ratio of the quantity of oil deposited (Q_{deposited}) expressed in μg of silicone/g of hair to the quantity of silicone used in the shampoo to treat the hair, expressed in μg of silicone/g of hair.

A composition containing an oil and a mixture consisting of an amphoteric surfactant and an alkyl polyglucoside makes it possible to obtain an improvement in the deposition of oil of greater than 30% relative to a composition comprising an oil and either an amphoteric surfactant or an alkyl polyglucoside. Preferably, this improvement is between 30% and 150% and preferably between 45% and 130%.

The present invention is based on the demonstration of a synergy of action between the amphoteric surfactant and the alkyl polyglucoside as regards the deposition of oil on hair.

In the present application, unless otherwise stated, the quantities are given as weight of active substance, by contrast to quantities given as weight of substance as it is.

Advantageously, the abovementioned composition comprises an amphoteric surfactant (true amphoteric compound comprising an ionic group and a potentially ionic group of opposite charge, or a zwitterionic group simultaneously comprising two opposite charges), chosen from the group consisting of alkyl amphoacetates, alkyl amphodiacetates, sultaines, amphosulfonates, betaines, alkyl amphopropionates and alkyl amphodipropionates, and mixtures thereof.

Among the preferred amphoteric surfactants, the following products may be mentioned in particular:
the alkyl amphoacetates marketed by the company Rhodia under the references Miranol® Ultra L-32 (sodium lauroamphoacetate) and Miranol® Ultra C-32 (sodium cocoamphoacetate);
the alkyl amphodiacetates marketed by the company Rhodia under the references Miranol® C2M Conc NP (disodium cocoamphodiacetate), Miranol® BM CONC and Miranol® H2M CONC (disodium lauroamphodiacetate);
the sultaines, in particular the hydroxysultaines, for example marketed by the company Rhodia under the trade references Mackam® CBS-50G (cocamidopropyl hydroxysultaine), Mackam® CBS (cocamidopropyl hydroxysultaine), Mackam® LSB-50 (lauramidopropyl hydroxysultaine), Mackam® 50-SB (cocamidopropyl hydroxysultaine) and Mackam® LHS (lauryl hydroxysultaine);
the amphosulfonates marketed by the company Rhodia under the trade references Miranol® CS and Miranol® CSE (Sodium Cocoamphohydroxypropyl Sulfonate) and Mackam® LS (Sodium Lauroamphohydroxypropyl Sulfonate);
the betaines, in particular the carboxybetaines and the amidoalkylbetaines, marketed by the company Rhodia under the trade references Mirataine® BB (lauramidopropylbetaine), Mirataine® BB FLA and Mackam® CB-35 (coco-betaine), Mackam® LAB (lauryl betaine), Mirataine® BET C-30 and Mackam® 50UL (cocamidopropyl betaine), Mirataine® BET O-30 (Oleamidopropyl betaine), marketed by the company Evonik under the trade reference TEGO® betain F (cocamidopropyl betaine);
the alkyl amphopropionates marketed by the company Rhodia under the trade reference Mackam® CSF-CG (sodium cocoamphopropionate); and
the alkyl amphodipropionates marketed by the company Rhodia under the trade references Miranol® C2M-SF Conc, Mackam® 2CSF-70 or Mackam® 2CSF-40 CG (disodium cocoamphodipropionate), Mackam® 168L (sodium lauriminodipropionate).

As alkyl polyglucosides, use is preferably made of those containing an alkyl group comprising from 6 to 30 carbon atoms, and preferably from 8 to 16 carbon atoms, and containing a hydrophilic group (glucoside) preferably comprising 1.2 to 3 saccharide units. As alkyl polyglucosides, mention may be made of alkyl glucosides, and for example decyl glucoside (C9/C11-alkyl polyglucoside (1.4)) such as the product marketed under the name MYDOL 10® by the company Kao Chemicals, under the name PLANTAREN 2000 UP® by the company Cognis, under the name ORAMIX NS 10® by the company Seppic, or under the name MACKOL DG® by the company Rhodia; caprylyl/capryl glucoside such as the product marketed under the name ORAMIX CG 110® by the company Seppic; lauryl glucoside such as the products marketed under the names PLANTAREN 1200 N® and PLANTACARE 1200® by the company Cognis; and coco glucoside such as the product marketed under the name PLANTACARE 818/UP® by the company Cognis.

Preferably, the alkyl polyglucoside of the composition as defined above is chosen from the group of alkyl glucosides and more particularly from the group consisting of cocoglucoside, decyl glucoside and lauryl glucoside, and mixtures thereof.

An advantageous use according to the present invention is characterized in that the oil present in the abovementioned composition is chosen from synthetic oils and vegetable oils.

Such oils may be chosen from alkyl monoglycerides, alkyl diglycerides, triglycerides such as the oils extracted from plants and vegetables (palm, copra, cottonseed, soya, sunflower, olive, grapeseed, sesame, peanut, castor, avocado, jojoba and apricot oils, and the like) or oils of animal origin (tallow oil, fish oils, and the like), derivatives of these oils such as hydrogenated oils, derivatives of lanolin, petrolatum, mineral oils or paraffin oils, perhydrosqualane, squalene, diols such as 1,2-dodecanediol, cetyl alcohol, stearyl alcohol, oleyl alcohol, fatty esters such as isopropyl palmitate, 2-ethylhexyl cocoate, myristyl myristate, esters of lactic acid, stearic acid, behenic acid, isostearic acid.

Finally, mention may be made of silicone oils (or polyorganosiloxanes), in particular chosen from polyalkyl siloxanes, polyaryl siloxanes, polyalkylaryl siloxanes, or their non-water-soluble functionalized derivatives, and their nonvolatile mixtures. The appropriate polyorganosiloxanes are in particular polydimethyl siloxanes (dimethicone), polyphenyl-dimethyl siloxanes, polydiethyl siloxanes, polymethyl-phenyl siloxanes, alkoxylated polyorganosiloxanes, amino-substituted polyorganosiloxanes, amido-substituted polyorganosiloxanes, and mixtures thereof. In a particularly preferred manner, use is made of polydimethyl siloxanes having a viscosity at least equal to 6×10⁵ mPa·s, at 25° C., and more preferably still those having a viscosity greater than 2×10⁶ mPa·s, at 25° C., such as Mirasil DM 500000® marketed by BlueStar Silicones.

The non-water-soluble and nonvolatile silicone or organopolysiloxane exists in a dispersed form in the cosmetic composition containing it. It is in the form of particles whose size may be chosen according to the nature of the cosmetic composition or the desired performance for said composition. In general, this size may vary from 0.02 to 70 microns. Preferably, this size is of the order of 0.05 to 70 microns, most particularly of the order of 0.05 to 30 microns.

These silicone particles may consist of mixtures of silicones whose presence may be due to the method of using the cosmetically advantageous silicone agent in the cosmetic composition. Thus, the cosmetically advantageous organopolysiloxanes may be dispersed or solubilized beforehand in volatile or nonvolatile silicone derivatives of low viscosity and then emulsified in the cosmetic composition. Among these low-viscosity silicones, mention may be made of cyclic volatile silicones and polydimethyl siloxanes of low mass.

As polyorganosiloxanes, mention may also be made of functionalized silicone derivatives such as amine-containing derivatives, such as Mirasil ADM-E® marketed by the company Rhone-Poulenc, (directly used in the form of emulsions in the cosmetic composition or from preformed microemulsions).

Preferably, the oil present in the above-mentioned composition is chosen from vegetable oils, mineral oils and silicone oils.

Preferably, the abovementioned composition comprises from 0.1% to 10%, and preferably from 1% to 5%, by weight of oil relative to the total weight of said composition.

According to an advantageous embodiment, the abovementioned composition additionally comprises an anionic surfactant.

The anionic surfactants may be chosen from the following surfactants:
alkyl ester sulfonates, for example of formula R—CH(SO₃M)-CH₂COOR′, or alkyl ester sulfates, for example of formula R—CH(OSO₃M)-CH₂COOR′, where R represents a C₈-C₂₀, preferably C₁₀-C₁₆, alkyl radical, R′ a C₁-C₆, preferably C₁-C₃, alkyl radical and M an alkaline-earth cation, for example sodium, or the ammonium cation. Mention may be made most particularly of methyl ester sulfonates whose R radical is a C₁₄-C₁₆;
alkyl, more particularly C₉-C₂₀ alkyl, benzenesulfonates, primary or secondary alkyl, in particular C₈-C₂₂ alkyl, sulfonates, alkyl glycerol sulfonates;
alkyl sulfates, for example of formula ROSO₃M, where R represents a C₁₀-C₂₄, preferably C₁₂-C₂₀, alkyl or hydroxyalkyl radical; M a cation having the same definition as above;
alkyl ether sulfates, for example of formula RO(OA)ₙSO₃M, where R represents a C₁₀-C₂₄, preferably C₁₂-C₂₀, alkyl or hydroxyalkyl radical; OA representing an ethoxylated and/or propoxylated group; representing a cation having the same definition as above, n generally varying from 1 to 4, such as for example lauryl ether sulfate with n=2;
alkyl amide sulfates, for example of formula RCONHR′OSO₃M, where R represents a C₂-C₂₂, preferably C₆-C₂₀, alkyl radical, R′ a C₂-C₃ alkyl radical, M representing a cation having the same definition as above, and their polyalkoxylated (ethoxylated and/or propoxylated) derivatives (alkyl amidoether sulfates);
salts of saturated or unsaturated fatty acids, for example such as those as C₈-C₂₄, preferably as C₁₄-C₂₀, and of an alkaline-earth cation, N-acyl N-alkyl taurates, alkyl isethionates, alkyl succinamates and alkyl sulfosuccinates, monoesters or diesters of sulfosuccinates, N-acyl sarcosinates, polyethoxy-carboxylates;
mono- and diester phosphates, for example having the following formula: (RO)ₓP(═O)(OM)ₓ′, where represents an alkyl, alkylaryl, arylalkyl or aryl radical, which are optionally polyalkoxylated, x and x′ being equal to 1 or 2, provided that the sum of x and x′ is equal to 3, M representing an alkaline-earth cation.

Preferably, the anionic surfactant present in the abovementioned composition is chosen from alkyl sulfates and alkyl ether sulfates.

In a particularly preferred manner, sodium lauryl ether sulfate (SLES) is used as anionic surfactant.

Preferably, the abovementioned composition comprises from 5% to 25%, and preferably from 5% to 15%, by weight of anionic surfactant relative to the total weight of said composition.

According to an advantageous embodiment, the abovementioned composition additionally comprises a cationic or amphoteric polymer.

Among these cationic or amphoteric polymers, the following compounds may be mentioned in particular: Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-43, Polyquaternium-44, Polyquaternium-45, Polyquaternium-46, Polyquaternium-47, Polyquaternium-48, Polyquaternium-49, Polyquaternium-50, Polyquaternium-52, Polyquaternium-53, Polyquaternium-54, Polyquaternium-55, Polyquaternium-56, Polyquaternium-57, Polyquaternium-58, Polyquaternium-59, Polyquaternium-60, Polyquaternium-63, Polyquaternium-64, Polyquaternium-65, Polyquaternium-66, Polyquaternium-67, Polyquaternium-70, Polyquaternium-73, Polyquaternium-74, Polyquaternium-75, Polyquaternium-76, Polyquaternium-85, Polyquaternium-86, Polybeta-Alanine, Polyepsilon-Lysine, Polylysine, PEG-8/SMDI copolymer, PPG-12/SMDI copolymer, PPG-51/SMDI copolymer, PPG-7/Succinic Acid copolymer, IPDI/PEG-15 Cocamine copolymer, IPDI/PEG-15 Cocamine copolymer dimer dilinoleate, IPDI/PEG-15 Soyamine copolymer, IPDI/PEG-15 Soyamine Oxide copolymer, IPDI/PEG-15 Soyethonium Ethosulfate copolymer, Polyquaternium-4/Hydroxypropyl starch copolymer, cassia hydroxypropyltrimonium chloride, chitosan hydroxypropyltrimonium chloride, dextran hydroxypropyltrimonium chloride, galactoarabinan hydroxypropyltrimonium chloride, ginseng hydroxypropyltrimonium chloride, guar hydroxy-propyltrimonium chloride, hydroxypropyl guar hydroxy-propyltrimonium chloride, carob hydroxypropyltrimonium chloride, starch hydroxypropyltrimonium chloride, hydroxypropyltrimonium hydrolyzed wheat starch, hydroxypropyltrimonium hydrolyzed corn starch, hydroxypropyl hydrolyzed starch PG-trimonium chloride, tamarindus indica hydroxypropyltrimonium chloride, polyacrylamidopropyltrimonium chloride, polymethacryl-amidopropyltrimonium chloride, polymethacrylamido-propyltrimonium methosulfate, propyltrimonium chloride methacrylamide/dimethylacrylamide copolymer, acryl-amide/ethalkonium chloride acrylate copolymer, acrylamide/ethyltrimonium chloride acrylate/ethalkonium chloride acrylate copolymer, acrylates/carbamate copolymer, adipic acid/methyl DEA polymer, diethylene glycol/DMAP Acrylamide/PEG-180/HDI copolymer, dihydroxyethyl tallowamine/IPDI copolymer, dimethyl-amine/ethylenediamine/epichlorohydrin copolymer, HEMA glucoside/ethylmethacrylate trimonium chloride copolymer, hydrolyzed wheat protein/PEG-20 acetate copolymer, hydrolyzed wheat protein/PVP copolymer, and ethyltrimonium chloride methacrylate/hydroxyethyl-acrylamide copolymer.

Preferably, the cationic or amphoteric polymer present in the abovementioned composition is chosen from the group consisting of cationic guars, cationic celluloses, cationic starches, cationic or amphoteric polymers comprising units derived from MAPTAC, DADMAC, DIQUAT or TRIQUAT, and optionally units derived from acrylamide and/or from vinylpyrrolidone and/or from acrylic acid.

Among the preferred cationic polymers according to the present invention, mention may be made in particular of the following commercial products: Jaguar® Excel (guar hydroxypropyltrimonium chloride) marketed by the company Rhodia, Polycare® 400 (polyquaternium-10) marketed by the company Rhodia, Ucare® JR-400 (polyquaternium-10) marketed by the company Dow-Amerchol and Jaguar® C-162 (hydroxypropyl guar hydroxypropyltrimonium chloride) marketed by the company Rhodia.

Preferably, the abovementioned composition comprises from 0.05% to 0.5%, and preferably from 0.1% to 0.4%, by weight of cationic or amphoteric polymer relative to the total weight of said composition.

According to another embodiment, the above-mentioned composition may also comprise a nonionic surfactant different from alkyl polyglycoside.

Preferably, the abovementioned composition comprises from 0% to 5% by weight of nonionic surfactant relative to the total weight of said composition.

As nonionic surfactant, mention may more particularly be made of ethoxylated fatty alcohols and in particular those derived from lauryl, cetylstearyl, stearyl, cetyl and oleocetyl alcohols. Sucroglycerides may also be used.

According to a preferred embodiment, these nonionic surfactants are chosen from optionally alkoxylated alkanolamides, amine oxides, optionally alkoxylated fatty alcohols, alkoxylated alkylphenols, fatty acid esters such as cocamide DEA, cocamide MIPA, PEG-5 cocamide MEA, lauramide DEA, lauramine oxide, cocamine oxide, stearamine oxide, stearamidopropylamine oxide, palmitamidopropylamine oxide, decylamine oxide, stearyl alcohol, sorbitan monolaurate, polysorbates, ethoxylated lauryl alcohols, and mixtures thereof.

According to another embodiment, the above-mentioned composition may also comprise an electrolyte.

Among the electrolytes, mention may be made of organic salts, inorganic salts and mixtures thereof. Among the salts, mention may be made of alkali or alkaline-earth metal, ammonium, amine or aminoalcohol salts. The preferred salts are chosen from sodium chloride, potassium chloride, lithium chloride, ammonium chloride, sodium sulfate, magnesium sulfate, sodium acetate, copper sulfate and sodium citrate.

According to a particularly preferred embodiment, the electrolyte is sodium chloride.

Preferably, the abovementioned composition comprises from 0% to 3%, and preferably from 0 or 0.5% to 1.5%, by weight of electrolyte relative to the total weight of said composition.

According to another embodiment, the above-mentioned composition may also comprise one or more additives conventionally used by a person skilled in the art in shampoo-type compositions. These additives are chosen in particular from the group consisting of preservatives, pearlescent agents, perfumes, colorants, pH regulators, stabilizing polymers, fixing polymers, rheology-modifying agents, sequestrants, cellulosic derivatives, antidandruff or antiseborrheic agents, organic or inorganic sunscreens, opacifying agents, proteins and protein derivatives, vitamins, provitamins, hydroxy acids, suspending agents, foam modifiers, humectants.

Preferably, the composition used in the context of the invention comprises from 0.5% to 10% by weight of the mixture consisting of an amphoteric surfactant and an alkyl polyglucoside.

In the mixture consisting of the amphoteric surfactant and the alkyl polyglucoside, the ratio between the weight of said surfactant and of said alkyl polyglucoside preferably varies from 4/1 to 1/4 and is preferably from 2/1 to 1/2, for example equal to 1.

In a particularly advantageous manner, the present invention relates to the use as defined above, characterized in that the composition is intended to be applied to damaged hair. Preferably, the present invention is characterized in that the hair to which the composition is applied is damaged hair. Preferably, the use of the composition is intended to increase the quantities of oil deposited on the damaged hair.

The damaged hair may be examined by microscopy, preferably with a scanning electron microscope. Typically, the damaged hair has the following characteristics: a separation, erosion or abrasion of the keratin scales, an increased porosity, bleached zones and/or split ends.

The damaged hair may also be characterized by a marked hydrophilic character, with an angle of contact of less than for example 90°.

The damage caused to the hair may be the consequence of various phenomena of a chemical or physical nature and the like.

The use of chemical products, of treatments such as permanent waving, dyeing treatments and bleaching/-lightening contribute toward greatly damaging the hair. Some of these chemical products cause swelling of the cuticle and lead to its deterioration with separation, erosion or abrasion of the keratin scales, exposing the cortex to future attacks to a greater or lesser degree. The treatments involving the use of alkaline solutions and of reducing or oxidizing agents induce degradation of cystine, with solubilization of the amino acids and peptides of keratin.

Mechanical treatments such as brushing, combing, drying, rubbing against various supports such as pillowcases, the use of hair accessories also damage the state of the hair fibers. Likewise, an intensive use of heating hair-styling instruments such as curling tongs and hair dryers greatly damages the hair.

Exposure to solar radiation and to air pollution also promotes deterioration of the state of the hair fibers.

The composition according to the invention may be applied to wet hair.

It has been observed that the compositions according to the present invention are particularly effective for improving the deposition of oil on damaged hair. This is particularly advantageous since it is known to a person skilled in the art that the deposition of oil, and in particular of silicone, is particularly difficult on damaged hair.

### EXPERIMENTAL PART

The protocol for preparing the formulas is as follows:

The required quantity of water was introduced into a beaker and the cationic polymer was slowly dispersed with stirring (300 revolutions per minute (rpm), paddle frame). Once the polymer was completely dispersed, the pH was adjusted to between 4 and 5 for the cationic guars (Jaguar Excel, Jaguar C-162) with the aid of a 10% citric acid solution and above 10 for the PQ-10 (Ucare JR-400, Polyquat 400KC) with the aid of a 20% sodium hydroxide solution, in order to promote their hydration. Once the solution is homogeneous, the amphoteric surfactant(s) was (were) added if necessary, followed by the nonionic surfactant(s), such as the alkyl polyglucosides. The anionic surfactant(s) was (were) then added. After stirring for 15 minutes, the silicone emulsion was added with stirring at 100 rpm. Once the solution is homogeneous, the preservative was added, the solution was adjusted to a pH of 5.5±0.2 with the aid of a 15% citric acid solution, and then the salt was added.

### Example 1

### Preparation of the Formulation F1 Based on Jaguar® Excel Comprising an Amphoteric Surfactant and an Alkyl Polyglucoside

A formulation F1 having the following composition was prepared:

| | | % active |
|---|---|---|
| Ingredient | | substance |
| Anionic surfactant | Empicol ESB 3M (Huntsman) | 12.0 |
| Amphoteric surfactant | Miranol ® Ultra C-32 (Rhodia) | 2.0 |
| Alkyl polyglucoside | Plantacare ® 818 (Cognis) | 2.0 |
| Cationic polymer | Jaguar ® Excel (Rhodia) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

### Comparative Examples 1 and 1bis

### Preparation of the Formulations F1′ and F″1 based on Jaguar® Excel

A formulation F′1 (with no alkyl polyglucoside) having the following composition was prepared:

| | | % active |
|---|---|---|
| Ingredient | | substance |
| \Anionic surfactant | Empicol ESB 3M (Huntsman) | 12.0 |
| Amphoteric surfactant | Miranol ® Ultra C-32 (Rhodia) | 4.0 |
| Alkyl polyglucoside | | — |
| Cationic polymer | Jaguar ® Excel (Rhodia) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

A formulation F″1 (with no amphoteric surfactant) having the following composition was prepared:

| | | % active |
|---|---|---|
| Ingredient | | substance |
| Anionic surfactant | Empicol ESB 3M (Huntsman) | 12.0 |
| Amphoteric surfactant | | — |
| Alkyl polyglucoside | Plantacare ® 818 (Cognis) | 4.0 |
| Cationic polymer | Jaguar ® Excel (Rhodia) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

### Example 2

### Preparation of the Formulation F2 Based on Jaguar® Excel Comprising an Amphoteric Surfactant and an Alkyl Polyglucoside

A formulation F2 having the following composition was prepared:

| | | % active |
|---|---|---|
| Ingredient | | substance |
| Anionic surfactant | Empicol ESB 3M (Huntsman) | 12.0 |
| Amphoteric surfactant | Miranol ® Ultra L-32 (Rhodia) | 2.0 |
| Alkyl polyglucoside | Plantacare ® 818 (Cognis) | 2.0 |
| Cationic polymer | Jaguar ® Excel (Rhodia) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

### Comparative Example 2

### Preparation of the Formulation F′2 Based on Jaguar® Excel

A formulation F′2 (with no alkyl polyglucoside) having the following composition was prepared:

| | | % active |
|---|---|---|
| Ingredient | | substance |
| Anionic surfactant | Empicol ESB 3M (Huntsman) | 12.0 |
| Amphoteric surfactant | Miranol ® Ultra L-32 (Rhodia) | 4.0 |
| Alkyl polyglucoside | | — |
| Cationic polymer | Jaguar ® Excel (Rhodia) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

### Example 3

### Preparation of the Formulation F3 Based on PQ-10 Comprising an Amphoteric Surfactant and an Alkyl Polyglucoside

A formulation F3 having the following composition was prepared:

| | | % active |
|---|---|---|
| Ingredient | | substance |
| Anionic surfactant | Empicol ESB 3M (Huntsman) | 12.0 |
| Amphoteric surfactant | Miranol ® Ultra L-32 (Rhodia) | 2.0 |
| Alkyl polyglucoside | Mackol ® DG (Rhodia) | 2.0 |
| Cationic polymer | Polycare ® 400 (Rhodia) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

### Comparative Examples 3 and 3bis

### Preparation of the Formulations F′3 and F″3 based on PQ-10

A formulation F′3 (with no alkyl polyglucoside) having the following composition was prepared:

| | | % active |
|---|---|---|
| Ingredient | | substance |
| Anionic surfactant | Empicol ESB 3M (Huntsman) | 12.0 |
| Amphoteric surfactant | Miranol ® Ultra L-32 (Rhodia) | 4.0 |
| Alkyl polyglucoside | | — |
| Cationic polymer | Polycare ® 400 (Rhodia) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

A formulation F″3 (with no amphoteric surfactant) having the following composition was prepared:

| | | % active |
|---|---|---|
| Ingredient | | substance |
| Anionic surfactant | Empicol ESB 3M (Huntsman) | 12.0 |
| Amphoteric surfactant | | — |
| Alkyl polyglucoside | Mackol ® DG (Rhodia) | 4.0 |
| Cationic polymer | Polycare ® 400 (Rhodia) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

### Examples 4, 5 and 6

### Preparation of the Formulations F4, F5 and F6 based on Ucare® JR-400 Comprising an Amphoteric Surfactant and an Alkyl Polyglucoside

A formulation F4 having the following composition was prepared:

| | | % active |
|---|---|---|
| Ingredient | | substance |
| Anionic surfactant | Empicol ESB 3M (Huntsman) | 12.0 |
| Amphoteric surfactant | Miranol ® Ultra C-32 (Rhodia) | 2.0 |
| Alkyl polyglucoside | Plantacare ® 818 (Cognis) | 2.0 |
| Cationic polymer | Ucare ® JR-400 (Dow-Amerchol) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

A formulation F5 having the following composition was prepared:

| | | % active |
|---|---|---|
| Ingredient | | substance |
| Anionic surfactant | Empicol ESB 3M (Huntsman) | 12.0 |
| Amphoteric surfactant | Miranol ® Ultra L-32 (Rhodia) | 2.0 |
| Alkyl polyglucoside | Plantacare ® 818 (Cognis) | 2.0 |
| Cationic polymer | Ucare ® JR-400 (Dow-Amerchol) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

A formulation F6 having the following composition was prepared:

| | | % active |
|---|---|---|
| Ingredient | | substance |
| Anionic surfactant | Empicol ESB 3M (Huntsman) | 12.0 |
| Amphoteric surfactant | TEGO betain F (Evonik) | 2.0 |
| Alkyl polyglucoside | Plantacare ® 818 (Cognis) | 2.0 |
| Cationic polymer | Ucare ® JR-400 (Dow-Amerchol) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

### Comparative Examples 4, 5 and 6

### Preparation of the Formulations F′4, F′5 and F′6 Based on Ucare® JR-400

A formulation F′4 (with no alkyl polyglucoside) having the following composition was prepared:

| | | % active |
|---|---|---|
| Ingredient | | substance |
| Anionic surfactant | Empicol ESB 3M (Huntsman) | 12.0 |
| Amphoteric surfactant | Miranol ® Ultra C-32 (Rhodia) | 4.0 |
| Alkyl polyglucoside | | |
| Cationic polymer | Ucare ® JR-400 (Dow-Amerchol) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

A formulation F′5 (with no alkyl polyglucoside) having the following composition was prepared:

| | | % active |
|---|---|---|
| Ingredient | | substance |
| Anionic surfactant | Empicol ESB 3M (Huntsman) | 12.0 |
| Amphoteric surfactant | Miranol ® Ultra L-32 (Rhodia) | 4.0 |
| Alkyl polyglucoside | | |
| Cationic polymer | Ucare ® JR-400 (Dow-Amerchol) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

A formulation F′6 (with no alkyl polyglucoside) having the following composition was prepared:

| | | % active |
|---|---|---|
| Ingredient | | substance |
| Anionic surfactant | Empicol ESB 3M (Huntsman) | 12.0 |
| Amphoteric surfactant | TEGO betain F (Evonik) | 4.0 |
| Alkyl polyglucoside | | |
| Cationic polymer | Ucare ® JR-400 (Dow-Amerchol) | 0.2 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

### Example 7

### Preparation of the Formulation F7 Based on Jaguar® C-162 Comprising an Amphoteric Surfactant and an Alkyl Polyglucoside

A formulation F7 having the following composition was prepared:

| | | % active |
|---|---|---|
| Ingredient | | substance |
| Anionic surfactant | Empicol ESB 3M (Huntsman) | 10.0 |
| Amphoteric surfactant | Miranol ® Ultra C-32 (Rhodia) | 1.5 |
| Alkyl polyglucoside | Mackol ® DG (Rhodia) | 1.5 |
| Cationic polymer | Jaguar ® C-162 (Rhodia) | 0.3 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

### Comparative Examples 7 and 7bis

### Preparation of the Formulations F′7 and F″7 Based on Jaguar® C-162

A formulation F′7 (with no alkyl polyglucoside) having the following composition was prepared:

| | | % active |
|---|---|---|
| Ingredient | | substance |
| Anionic surfactant | Empicol ESB 3M (Huntsman) | 10.0 |
| Amphoteric surfactant | Miranol ® Ultra C-32 (Rhodia) | 3.0 |
| Alkyl polyglucoside | | — |
| Cationic polymer | Jaguar ® C-162 (Rhodia) | 0.3 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

A formulation F″7 (with no amphoteric surfactant) having the following composition was prepared:

| | | % active |
|---|---|---|
| Ingredient | | substance |
| Anionic surfactant | Empicol ESB 3M (Huntsman) | 10.0 |
| Amphoteric surfactant | | — |
| Alkyl polyglucoside | Mackol ® DG (Rhodia) | 3.0 |
| Cationic polymer | Jaguar ® C-162 (Rhodia) | 0.3 |
| Silicone | Emulsion of dimethicone | 2.0 |
| | Mirasil DM500000 (Bluestar | |
| | Silicones) 0.6 μm | |
| Preservative | Kathon CG (Rohm & Haas) | 0.05 |
| pH regulator | Citric acid (15% solution) | q.s. pH 5.5 |
| Salt | Sodium chloride | 1.0 |
| | Demineralized water | q.s. 100 |

### Example 8

### Measurement of the Deposition of Silicone on Hair

Locks of natural brown hair and of bleached hair, referenced as follows in IHIP (International Hair Importers & Products Inc.), are used:
“Virgin Medium Brown Caucasian Hair”—4.5 g—15-20 cm in length
“Regular Bleached Caucasian Hair”—4.5 g—15-20 cm in length

The protocol for evaluating the quantity of deposited silicone comprises four steps: pretreatment of the locks with a 10% solution of active substance of sodium lauryl ether sulfate, treatment of the locks with the shampoo tested, extraction of silicone with tetrahydrofuran (THF) and assaying of the extracted silicone by GPC.

### Pretreatment of the Locks:

The hair locks are pretreated with a 10% SLES solution of active substance, followed by rinsing under water for 1 min. The protocol is as follows: set the flow rate of water at 150 ml/s and the temperature at 38° C., wet the lock for 1 minute by placing it under the jet of water, deposit 3 ml of a 10% SLES solution of active substance on the entire length of the lock and massage over the entire length of the lock for 1 minute, rinse the lock by placing it under the jet of water for 1 minute.

### Treatment of the Locks:

Precisely weigh about 450 mg of shampoo in a cup. Note the precise mass, mₛₕₐₘₚₒₒ. Wind the lock around the finger and recover the shampoo. Massage (cause to foam) for 45 s. Rinse for 30 seconds (flow rate: 150 ml/s; temperature: 38° C.). Drain the lock by passing between the index finger and the middle finger. Allow the locks to dry over night in a controlled-environment room (21° C., 50% RH).

### Extraction of Silicone:

For each lock, tare a 250 ml polyethylene bottle. Introduce the lock into the bottle while keeping the glue part outside the bottle. Cut the lock short just below the glue and note the exact mass of hair introduced into the bottle, mₕₐᵢᵣ (g). Introduce into the bottle about 100 ml of THF, and close the bottle. Place the bottles on the shaking table and leave to shake for 24 hours at 200 revolutions/minute. Under the safety cabinet, with gloves, pour the THF extraction solution into a 150 ml crystallizing dish. Leave to evaporate under the safety cabinet (maximum speed of aspiration) for 24 hours.

### Assaying of the Extracted Silicone:

Tare the crystallizing dish surmounted by a watch glass. Under the safety cabinet, introduce about 4 ml of THF into the crystallizing dish. Using a spatula, scrape the bottom of the crystallizing dish and its walls in order to solubilize all the deposit. Weigh the crystallizing dish again surmounted by the watch glass and note the exact mass of THF introduced, m_{THF} (g). Using a syringe, transfer the dimethicone solution into a 2 ml bottle and then close the bottle. Assay the dimethicone concentration in the bottle by GPC, ELSD (evaporative light scattering detector).

The quantity of silicone deposited on the hair, Q_{deposited}, expressed in μg of silicone/g of hair is calculated by the equation (1):

$${Q}_{deposited} ( µ g of silicone / g of hair ) = \frac{{C}_{silicone} ( ppm ) \cdot {m}_{THF} ( g )}{{m}_{hair} ( g )} \qquad ( 1 )$$

where C_{silicone} is the concentration of silicone extracted by GPC in the 2 ml bottle, expressed in ppm (μg of silicone/g of THF).

The quantity of silicone used in the shampoo to treat the hair is calculated by the equation (2):

Q_{used}(μg of silicone/g of hair)=

$$\frac{{}_{}^{m}{shampoo}{}_{}^{( µ g )} \cdot {}^{Φ} silicone}{{}^{m} hair ( g )} \qquad ( 2 )$$

where Φ_{silicone} is the mass fraction of silicone active substance in the shampoo.

From these results, the deposition yield (3) is deduced

$$Y ( \% ) = \frac{{}_{}^{c}{silicone}{}_{}^{( ppm )} \cdot {}^{m} THF ( g )}{{}_{}^{m}{shampoo}{}_{}^{( µ g )} \cdot {}^{Φ} silicone} \qquad ( 3 )$$

A minimum of two assays (that is two locks) is carried out for each formulation evaluated, and the mean of the two assays is determined.

The yield of deposition of silicone was measured for the abovementioned formulations, namely the formulations F1, F2, F3, F4, F5, F6 and F7 according to the invention and the formulations for comparison F′1, F″1, F′2, F″2, F′3, F″3, F′4, F′S, F′6, F′7 and F″7.

### Example 8.1

### Formulations F1, F′1 and F″1

The results obtained on healthy hair are the following:

| | Formulation | | |
|---|---|---|---|
| | F1 | F′1 | F″1 |
| % deposition of silicone | 36.2 | 25.2 | 22.8 |

It is therefore observed that the formulation F1 comprising an alkyl polyglucoside and an amphoteric surfactant allows an increased deposition of silicone compared with the formulations F′1 (comprising an amphoteric surfactant but no alkyl polyglucoside) and F″1 (comprising an alkyl polyglucoside but no amphoteric surfactant). This therefore indeed demonstrates the improving effect on the deposition of silicone of the alkyl polyglucoside/amphoteric surfactant combination.

### Example 8.2

### Formulations F2, F′2 and F″1

The results obtained on healthy hair are the following:

| | Formulation | | |
|---|---|---|---|
| | F2 | F′2 | F″1 |
| % deposition of silicone | 39.5 | 28.6 | 22.8 |

It is therefore observed that the formulation F2 comprising an alkyl polyglucoside and an amphoteric surfactant allows an increased deposition of silicone compared with the formulations F′2 (comprising an amphoteric surfactant but no alkyl polyglucoside) and F″1 (comprising an alkyl polyglucoside but no amphoteric surfactant). This therefore indeed demonstrates the improving effect on the deposition of silicone of the alkyl polyglucoside/amphoteric surfactant combination.

### Example 8.3

### Formulations F3, F′3 and F″3

The results obtained on healthy hair are the following:

| | Formulation | | |
|---|---|---|---|
| | F3 | F′3 | F″3 |
| % deposition of silicone | 28.8 | 0 | 12.2 |

It is therefore observed that the formulation F3 comprising an alkyl polyglucoside and an amphoteric surfactant allows an increased deposition of silicone compared with the formulations F′3 (comprising an amphoteric surfactant but no alkyl polyglucoside) and F″3 (comprising an alkyl polyglucoside but no amphoteric surfactant). This therefore indeed demonstrates the improving effect on the deposition of silicone of the alkyl polyglucoside/amphoteric surfactant combination.

### Example 8.4

### Formulations F4, F5, F6, F′4, F′5 and F′6

The results obtained on healthy hair are the following:

Formulation

| | F4 | F5 | F6 | F′4 | F′5 | F′6 |
|---|---|---|---|---|---|---|
| % deposition of silicone | 2.1 | 3.6 | 4.1 | 0 | 0 | 0 |

It is therefore observed that the formulations F4, F5 and F6 comprising an alkyl polyglucoside and an amphoteric surfactant allow an increased deposition of silicone compared with the formulations F′4, F′S and F′6 (comprising an amphoteric surfactant but no alkyl polyglucoside). This therefore indeed demonstrates the improving effect on the deposition of silicone of the alkyl polyglucoside/amphoteric surfactant combination.

### Example 8.5

### Formulations F7, F′7 and F″7

The results obtained on healthy hair are the following:

| | Formulation | | |
|---|---|---|---|
| | F7 | F′7 | F″7 |
| % deposition of silicone | 33.6 | 19.1 | 20.6 |

The results obtained on damaged hair are the following:

| | Formulation | | |
|---|---|---|---|
| | F7 | F′7 | F″7 |
| % deposition of silicone | 5.6 | 0 | 0 |

It is therefore observed that the formulation F7 comprising an alkyl polyglucoside and an amphoteric surfactant allows an increased deposition of silicone compared with the formulations F′7 (comprising an amphoteric surfactant but no alkyl polyglucoside) and F″7 (comprising an alkyl polyglucoside but no amphoteric surfactant). This therefore indeed demonstrates the improving effect on the deposition of silicone of the alkyl polyglucoside/amphoteric surfactant combination, both on healthy hair and on damaged hair.

1. A composition, intended to be applied to hair, the composition comprising an oil and a mixture comprising an amphoteric surfactant and an alkyl polyglucoside to increase a quality of oil deposited on the hair.

2. The composition as described in claim 1, wherein the amphoteric surfactant is selected from the group consisting of an alkyl amphoacetate, an alkyl amphodiacetate, an alkyl amphopropionate, an alkyl amphodipropionate, a sultaine, an amphosulfonates and a betaine.

3. The composition as described in claim 1, wherein the alkyl polyglucoside is selected from the group consisting of a coco glucoside, a decyl glucoside and a lauryl glucoside, and a mixture thereof.

4. The composition as described in claim 1, wherein the oil is selected from the group consisting of a vegetable oil, a mineral oil and a silicone oil.

5. The composition as described in claim 1, wherein the composition additionally comprises an anionic surfactant, selected from the group consisting of an alkyl sulfate and an alkyl ether sulfate.

6. The composition as described in claim 1, wherein the composition additionally comprises a cationic or an amphoteric polymer, selected from the group consisting of a cationic guar, a cationic cellulose, a cationic starch, a cationic or an amphoteric polymer comprising units derived from MAPTAC, DADMAC, DIQUAT or TRIQUAT, and optionally units derived from acrylamide and/or from vinylpyrrolidone and/or from an acrylic acid.

7. The composition as described in claim 1, wherein the composition also comprises a nonionic surfactant different from alkyl polyglycoside.

8. The composition as described in claim 1, wherein the composition also comprises an electrolyte.

9. The composition as described in claim 1, wherein the composition also comprises one or more additives selected from the group consisting of a preservative, a pearlescent agent, a perfume, a colorant, a pH regulator, a stabilizing polymer, a fixing polymer, a rheology-modifying agent, a sequestrant, a cellulosic derivative, an antidandruff or an antiseborrheic agent, an organic or an inorganic sunscreen, an opacifying agent, a protein and a protein derivative, a vitamin, a provitamin, a hydroxy acid, a suspending agents, a foam modifier, and a humectant.

10. The composition as described in claim 1, wherein the composition comprises from 0.5% to 10% by weight of the mixture comprising an amphoteric surfactant and an alkyl polyglucoside, with a weight ratio of amphoteric surfactant to alkyl polyglucoside optionally of from 4/1 to 1/4.

11. The composition as described in claim 1, wherein the hair to which the composition is applied is damaged hair.

12. A method of increasing a quantity of oil deposited on hair, the method comprising administering a composition to the hair wherein the composition comprises an oil and a mixture comprising an amphoteric surfactant and an alkylpolyglucoside in an amount effective to increase the quantity of oil deposited on the hair.

13. The method as described in claim 12, wherein the amphoteric surfactant is selected from the group consisting of an alkyl amphoacetate, an alkyl amphodiacetate, an alkyl amphopropionate, an alkyl amphodipropionate, a sultaine, an amphosulfonates and a betaine.

14. The method as described in claim 12, wherein the alkyl polyglucoside is selected from the group consisting of a coco glucoside, a decyl glucoside and a lauryl glucoside, and a mixture thereof.

15. The method as described in claim 12, wherein the oil is selected from the group consisting of a vegetable oil, a mineral oil and a silicone oil.

16. The method as described in claim 12, wherein the composition additionally comprises an anionic surfactant, selected from the group consisting of an alkyl sulfate and an alkyl ether sulfate.

17. The method as described in claim 12, wherein the composition additionally comprises a cationic or an amphoteric polymer, selected from the group consisting of a cationic guar, a cationic cellulose, a cationic starch, a cationic or an amphoteric polymer comprising units derived from MAPTAC, DADMAC, DIQUAT or TRIQUAT, and optionally units derived from acrylamide and/or from vinylpyrrolidone and/or from an acrylic acid.

18. The method as described in claim 12, wherein the composition also comprises a nonionic surfactant different from alkyl polyglycoside.

19. The method as described in claim 12, wherein the composition also comprises an electrolyte.

20. The method as described in claim 12, wherein the composition also comprises one or more additives selected from the group consisting of a preservative, a pearlescent agent, a perfume, a colorant, a pH regulator, a stabilizing polymer, a fixing polymer, a rheology-modifying agent, a sequestrant, a cellulosic derivative, an antidandruff or an antiseborrheic agent, an organic or an inorganic sunscreen, an opacifying agent, a protein and a protein derivative, a vitamin, a provitamin, a hydroxy acid, a suspending agents, a foam modifier and a humectant.

21. The composition as described in claim 12, wherein the composition comprises from 0.5% to 10% by weight of the mixture comprising of an amphoteric surfactant and an alkyl polyglucoside, with a weight ratio of amphoteric surfactant to alkyl polyglucoside optionally of from 4/1 to 1/4.

22. The composition as described in claim 12, wherein the hair to which the composition is applied is damaged hair.
